(19) United States
(12) Patent Application Publication
(10) Pub. No.: US 2011/0065815 A1
(43) Pub. Date: Mar. 17, 2011

(54) METHOD AND APPARATUS FOR PRODUCTION OF HYDROCARBON FROM BIOMASS

(76) Inventors: Masayasu Sakai, Nagasaki (JP); Tadashi Yokoi, Chiba-shi (JP)

(21) Appl. No.: 12/673,771

(22) PCT Filed: Aug. 13, 2008
(86) PCT No.: PCT/JP2008/064524

§ 371 Date: Dec. 2, 2010

(30) Foreign Application Priority Data
Aug. 17, 2007 (JP) 2007-212822

(51) Int. Cl. C07C 27/06; B01J 19/00

(52) U.S. Cl. 518/704; 422/187

(57) ABSTRACT

Disclosed is an apparatus for producing a hydrocarbon from a biomass such as a glass or wood, which comprises: a biomass gasification unit (101) for feeding a raw material biomass (MB) and an overheated water vapor (S) for gasifying the biomass and a fuel biomass (FB) and air (A) for burning the fuel biomass, thereby producing a mixed gas (G) mainly composed of hydrogen and carbon monoxide; a clean-up means (201) for purifying the mixed gas (G); a gas tank (301) for storing the cleaned-up mixed gas (G) temporarily; a pressurizing pump (401) for pressurizing the mixed gas (G); and a hydrocarbon synthesis unit (501) for converting the pressurized mixed gas (G) into a hydrocarbon.

### TECHNICAL FIELD

The present invention relates to a method and an apparatus for carrying out synthesis of a hydrocarbon series liquid or a gas as a product using hydrogen and carbon monoxide, which are generated by use of a biomass such as grass or a tree as a raw material, as a reactant and especially relates to a producing method and an apparatus of hydrocarbon utilizing Fischer-Tropsch method (hereinafter referred to as FT method) as a synthesis method of the hydrocarbon.

### BACKGROUND ART

Conventionally, synthesis of a petroleum alternative synthetic fuel by the FT method for synthesizing liquidized hydrocarbon has been carried out by catalytic reaction between hydrogen and carbon monoxide, which are obtained by partially burning a natural gas or resolving water vapor by coal under a high temperature and high pressure condition. According to this FT method, in a case where petroleum resources are drained or price of the petroleum is elevated, petroleum alternative fuel can be temporarily synthesized from a natural gas or coal by the FT method. Therefore, various studies, improvement, and modification of the method have been conducted.

However, there are problems that a synthesis method and a synthesis apparatus of a hydrocarbon fuel by the conventional FT method require very high pressure and size of the apparatus becomes large due to the demand for the high pressure.

Meanwhile, along with the strong demand for usage of recyclable resources these days, necessity of usage of a biomass energy has been strongly recognized. This is because there is high expectation to utilization of the biomass energy from a viewpoint of possibility of recycling or possibility of mass production while there is still a big problem in direct utilization of the natural gas or coal or synthetic fuel from the natural gas or coal by the above-mentioned FT method as a petroleum alternative fuel from a viewpoint of global warming caused by emission of carbon dioxide or recyclability, although certain usefulness thereof is recognized.

To respond to such expectation, the inventors of the present invention have proceeded development of a biomass gasification unit or an apparatus for generation of high-temperature combustion gas to generate hydrogen and carbon monoxide using a biomass as a raw material or a fuel as disclosed in the Patent Document 1 and the Patent Document 2.

However, because it is difficult to obtain hydrogen and carbon monoxide both quantitatively and qualitatively from the conventional biomass gasification unit in the Patent Document 1 or the like, there has been no method and apparatus to synthesize liquid or gas hydrocarbon fuel by use of the FT method while using hydrogen and carbon monoxide, which is obtained by using biomass as a raw material, as a reactant.

### Patent Document 1: Japanese Unexamined Patent Application Publication No. 2005-105285

### Patent Document 2: Japanese Unexamined Patent Application Publication No. 2006-300501

The present invention has been made in consideration of the above-mentioned condition and a first purpose thereof is to provide a method for producing hydrocarbon from a biomass which uses hydrogen and carbon monoxide, which is generated by use of a biomass such as grass or a tree, as a reactant and enables to synthesize liquid or gas hydrocarbon fuel as a product with a high yield while being small-sized and low pressured. A second purpose of the present invention is to provide an apparatus for producing hydrocarbon from a biomass.

### SUMMARY OF INVENTION

### Technical Problem

To achieve the above-mentioned first purpose, a method for producing hydrocarbon from a biomass of the present invention is characterized by generating a mixed gas including hydrogen and carbon monoxide as main components thereof by heating powder or chipped raw material biomass to 800° C. or more and at the same time bringing the biomass into contact with water vapor of 800° C. or more and causing the mixed gas to be in contact with a predetermined catalyst under a predetermined temperature and a predetermined pressure to convert the mixed gas into hydrocarbon.

It is preferable that the pressure under which the mixed gas is brought into contact with the catalyst is lower than 3 MPa.

It is preferable that the catalyst to be in contact with the mixed gas has a single body or a compound of one selected from iron and copper or both of them as a basic catalyst and at the same time a single body or a compound of one or more substances selected from magnesium, calcium, cobalt, nickel, potassium, and sodium which is added as a backup catalyst or assistant catalyst, while one or more substances selected from zeolite, alumina, and silica are supported.

Catalytic reaction is a chemical reaction expressed by the following chemical equation formula.

CO+2H₂→n⁻¹(CH₂)ₙ+H₂O

It becomes possible to repeatedly carry out predetermined catalytic reaction to the mixed gas and to reduce the amount of the unreacted mixed gas while gradually converting the unreacted mixed gas into hydrocarbon if a route of bringing the mixed gas into contact with the catalyst to convert the gas into hydrocarbon by the predetermined catalytic reaction and bringing unreacted mixed gas remained in a previous step into contact with a catalyst equivalent to the aforementioned catalyst so that the gas is converted into hydrocarbon by the catalytic reaction is set to include predetermined number of steps.

Hydrogen obtained by electrolysis of water by the power of a recyclable energy other than a biomass is added to the mixed gas including hydrogen and carbon monoxide as its main components which are generated by heating the raw material biomass to 800° C. or more and at the same time bringing the biomass into contact with water vapor of 800° C. or more enables to improve yield of hydrocarbon per raw material biomass.

To achieve the second purpose, means taken in an apparatus for producing hydrocarbon from a biomass of the present invention is characterized by including a biomass gasification unit for generating a mixed gas including hydrogen and carbon monoxide as main components thereof using a biomass as a raw material and fuel, a pressurization means for pressurizing the mixed gas generated by the biomass gasification unit, a temperature adjustment means for adjusting the mixed gas to have an appropriate temperature, a catalyst for obtaining hydrocarbon as a product by causing a predetermined catalytic reaction using the mixed gas which is pressurized by the pressurization means while maintained in the appropriate temperature by the temperature adjustment means as a reactant, a reaction chamber in which the catalyst is provided and the mixed gas which is appropriately pressurized and has appropriate temperature is brought into contact with the catalyst to cause a predetermined catalytic reaction, a liquidization means for liquidizing the hydrocarbon generated by the catalytic reaction, and a collection means for collecting the liquidized hydrocarbon liquidized by the liquidization means.

It is preferable that the reaction chamber includes an inlet for supplying the mixed gas and an exhaust for discharging the hydrocarbon generated by the catalytic reaction in the reaction chamber and the unreacted mixed gas wherein the catalyst is provided on a route from the inlet to the exhaust.

The reaction chamber has the inlet for supplying the mixed gas in an upper part thereof, has the exhaust for discharging the hydrocarbon generated by the catalytic reaction of the mixed gas in the reaction chamber and the unreacted mixed gas in an lower part of the chamber, and the catalyst is provided on a route from the inlet to the exhaust. A plurality of the reaction chambers are provided, the inlet of the reaction chamber positioned at the uppermost stream connected the biomass gasification unit so that the mixed gas generated by the biomass gasification unit can be introduced, the exhaust of the reaction chamber positioned at the uppermost stream connected to the inlet of the reaction chamber positioned on immediate downstream, and hereinafter an exhaust of the reaction chamber positioned on immediate upper stream is connected to the inlet of the reaction chamber positioned on immediate downstream.

It is preferable that the liquidization means includes a liquidization chamber having the inlet on the upper stream side and an exhaust for discharging the unreacted mixed gas on the down stream side and an extraction port for extracting liquidized hydrocarbon on the down stream side and a cooling means for cooling down the liquidization chamber, wherein the inlet is connected to the exhaust of the reaction chamber positioned on the upper stream side of the liquidization chamber and the exhaust is connected to the inlet of the reaction chamber positioned on the down stream side of the liquidization chamber.

The collection means for collecting the liquidized hydrocarbon includes a pipeline connecting with each extraction ports of the plurality of liquidization chambers wherein the pipeline can be freely opened or closed by a valve provided on a route thereof.

The temperature adjustment means for adjusting temperature of the mixed gas to be the predetermined temperature before introduction of the mixed gas into the reaction chamber may intervene on the upper stream side of the inlet for supplying the mixed gas into the reaction chamber.

The reaction chamber may include an air inlet for supplying air controlled to have a predetermined temperature by a constant room temperature adjustment means and an air exhaust for emitting air and may be provided in a constant temperature room which is partitioned and surrounded by an heat insulating material.

It is preferable that the catalyst has a single body or a compound of one selected from iron and copper or both of them as a basic catalyst and at the same time a single body or a compound of one or more substances selected from magnesium, calcium, cobalt, nickel, potassium, and sodium is added as a backup catalyst or assistant catalyst thereto, while one or more substances selected from zeolite, alumina, and silica are supported.

The biomass gasification unit used for the present invention includes an heat insulating room which is partitioned and surrounded by an heat insulating wall material, a gasification reaction chamber having a raw material biomass introduction means partitioned and surrounded by a thermally conductive wall material in the heat insulating room for supplying the raw material biomass roughly crushed to have a diameter of approximately 2 cm or less inside and an overheated water vapor introduction means for supplying overheated water vapor inside, and a combustion high temperature gas generation unit for supplying a combustion high temperature gas in a space between the heat insulating room and the gasification reaction chamber, wherein the raw material biomass and the overheated water vapor introduced into the gasification reaction chamber are heated by the combustion high temperature gas supplied from the combustion high temperature gas generation unit to the space between the heat insulating room and the gasification reaction chamber via the thermally conductive wall material of the gasification reaction chamber to cause the raw material biomass and the overheated water vapor to react endothermically so that the mixed gas including hydrogen and carbon monoxide as main components thereof is generated.

The combustion high temperature gas generation unit generates a combustion high temperature gas of 800° C. or more by perfectly combusting a fuel biomass and supplies the generated combustion high temperature gas to the space between the heat insulating room and the gasification reaction chamber.

### BRIEF DESCRIPTION OF DRAWINGS

FIG. 1 is a schematic view showing configuration of the whole of an apparatus for producing hydrocarbon from a biomass.

FIG. 2 is a schematic view showing configuration of the whole of a biomass gasification unit.

FIG. 3 is a view showing a main part of the gasification unit of a biomass and also showing configuration of a unit for generating a mixed gas.

FIG. 4 is a view showing configuration of a combustion high temperature gas generation unit.

FIG. 5 is a view showing configuration of a hydrocarbon synthesis unit, a liquidization means, and a collection means.

### REFERENCE NUMERALS

1: An apparatus for producing hydrocarbon from a biomass;
101: Biomass gasification unit;
110 Heat insulating room;
111: Combustion high temperature gas inlet;
112: Combustion high temperature gas exhaust;
120: Gasification reaction chamber;
121: Porous body;
122: Ash discharging means;
123: Mixed gas exhaust means;
124: Gasification space;
125: Raw material biomass inlet;
126: Overheated water vapor inlet;
127: Mixed gas exhaust;
128: Ash exhaust;
130: Raw material biomass introduction means;
131: Screw feeder;
132: Hopper;
140: Overheated water vapor introduction means;
141: Boiler;
142: Fan motor;
143: Chimney;
150: Combustion high temperature gas generation unit;
151: Fire lattice;
152: Combustion furnace;
153: Air preheating device;
154: Upper combustion room;
155: Bottom combustion room;
156: Fuel biomass inlet;
157: First air inlet;
158: Second air inlet;
159: Third air inlet;
160: Combustion high temperature gas exit;
161: Ash pit;
201: Cleaning up means;
202: Heat exchanger;
203: Cyclone;
204: Water injector;
301: Gas tank;
302: Purified mixed gas inlet;
303: Mixed gas exit;
304: Inlet for reintroduction of mixed gas;
401: Pressurization pump;
501: Hydrocarbon synthesis unit;
510: Pressurized mixed gas inlet;
520: Temperature adjustment means;
521: Constant temperature room;
522: Constant temperature room temperature adjustment unit;
523: Temperature-controlled air inlet;
524: Temperature-controlled air exhaust;
525: High temperature gas introduction line;
526: Air introduction line;
527: Temperature-controlled air feeding line;
30a: First reaction chamber;
530b: Second reaction chamber;
530c: Third reaction chamber;
530d: Fourth reaction chamber;
530e: Fifth reaction chamber;
531: Catalyst;
532a: First mixed gas introduction line;
532b: Second mixed gas introduction line;
532c: Third mixed gas introduction line;
532d: Fourth mixed gas introduction line;
532e: Fifth mixed gas introduction line;
533a, 533b, 533c, 533d, 533e: Mixed gas inlets;
534a, 534b, 534c, 534d, 534e: Exhaust lines;
535a, 535b, 535c, 535d, 535e: Temperature adjustment units;
540: Liquidization means;
541a: First liquidization chamber;
541b: Second liquidization chamber;
541c: Third liquidization chamber;
541d: Fourth liquidization chamber;
541e: Fifth liquidization chamber;
542: Cooling water introduction line;
543: Cooling water discharging line;
544: Cooling bath;
545a, 545b, 545c, 545d, 545e: Introduction lines;
546a, 546b, 546c, 546d, 546e: Liquidized hydrocarbon exhausts;
547a, 547b, 547c, 547d: Cooled mixed gas exhausts;
547e: Final unreacted mixed gas exhaust;
548: Circulation line;
550: Collection means;
551a: First collection pipe;
551b: Second collection pipe;
551c: Third collection pipe;
551d: Fourth collection pipe;
551e: Fifth collection pipe;
552: Liquidized hydrocarbon extraction pipe;
553: Valve;
A, A1, A2, A3: Air;
B: Combustion high temperature gas;
FB: Fuel biomass;
G: Mixed gas;
MB: Raw material biomass;
S: Overheated water vapor;
TA: Temperature-controlled air; and
W: Cooling water

### BEST MODES FOR CARRYING OUT THE INVENTION

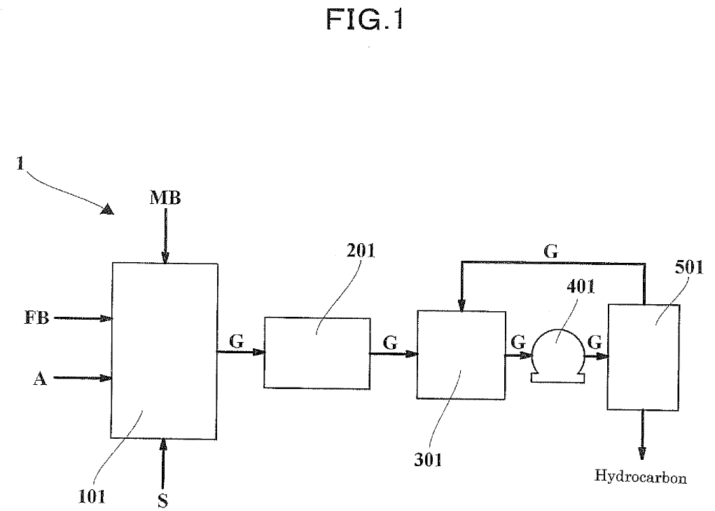

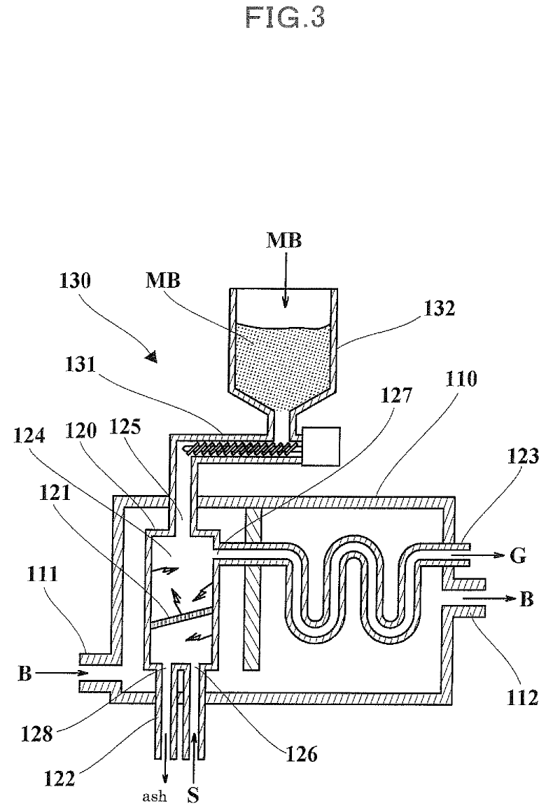

Hereinafter, a preferable embodiment of the present invention will be explained in detail with reference to FIGS. 1 to 5.

A method and an apparatus for producing hydrocarbon from a biomass of the present embodiment use a powder or chipped biomass as a raw material. The raw material biomass is heated to 800° C. or more and brought into contact with water vapor of 800° C. or more to generate a mixed gas including hydrogen and carbon monoxide as its main components. The mixed gas generated from biomass is used as a reactant. The reactant is converted into hydrocarbon by the FT method and liquidized to obtain a liquid hydrocarbon of synthetic fuel.

The method of the present embodiment for synthesizing liquid hydrocarbon from the mixed gas generated from biomass including hydrogen and carbon monoxide as its main components is to set the mixed gas to be between 150° C. and 300° C. and at the same time to pressurize the gas with less than 3 MPa of pressure to bring the gas into contact with a predetermined catalyst to cause a predetermined catalytic reaction as expressed by the above-mentioned chemical equation formula so that hydrogen and carbon monoxide are converted into gas phase hydrocarbon and heat exchange is carried out between refrigerant substances such as water or air to cool down the gas phase hydrocarbon to obtain liquid hydrocarbon.

The catalyst has a single body or a compound of one selected from iron and copper or both of them as a basic catalyst and at the same time a single body or a compound of one or more substances selected from magnesium, calcium, cobalt, nickel, potassium, and sodium is added as a backup catalyst or assistant catalyst thereto, while one or more substances selected from zeolite, alumina, and silica are supported.

As the catalyst, for example, a catalyst configured by combining a heretofore known FT catalyst and a solid acid catalyst such as zeolite may be used and in this case as the catalytic reaction, a mixed gas including hydrogen and carbon monoxide is reacted first on the FT catalyst to generate heavy hydrocarbon.

Subsequently, the heavy hydrocarbon is decomposed on the adjacent solid acid catalyst to be a lighter branch hydrocarbon. According to the catalyst having such a configuration, there are advantages such as it becomes possible to synthesize hydrocarbon from the mixed gas including hydrogen and carbon monoxide and moreover to automatically decompose and remove wax, which is accumulated on the FT catalyst to cause a problem before, so that slowing down of expansion of the mixed gas due to deactivation of the catalyst or to the wax can be suppressed.

Specifically, as the FT catalyst, a cobalt FT catalyst with required loading of cobalt can be obtained by impregnating cobalt nitrate into silica gel which was previously dried for two hours in the air at 200° C. by incipient wetness method, subsequently drying for 12 hours at 120° C. and then burning at 400° C. for two hours for preparation. Moreover, it is also possible to prepare an iron FT catalyst by solving predetermined amount of iron nitrate, copper nitrate, magnesium nitrate, and calcium nitrate in 500 mL of water and simultaneously mixing. The solution and sodium carbonate solution which was adjusted to 20 g/500 mL are dropped into 500 mL of water adjusted 60° C. and pH of 8 to generate precipitate, and then, after all the solutions are dropped, the solutions are further mixed for one hour, the precipitate is filtered, washed by distilled water, dried, and burnt at 400° C. for preparation.

Further, a composite catalyst configured by combining the above-mentioned zeolite and the FT catalyst can be prepared by preparing a sol precursor solution by use of tetraethyl orthosilicate, aluminum nitrate, tetrapropyl ammonium hydroxide, water, and ethanol while mixing previously prepared cobalt FT catalyst and zeolite and then putting the prepared solution into an autoclave for hydrothermal synthesis at 180° C. Moreover, it is possible to prepare the composite catalyst by mixing the previously prepared iron FT catalyst and zeolite and then pressurizing the mixture for 20 minutes by a uniaxial pressing machine with 600 kgf/cm² of pressure. Then, a cobalt series composite catalyst obtained by combining the cobalt FT catalyst and the zeolite catalyst, and an iron series composite catalyst obtained by combining the iron FT catalyst and the zeolite catalyst may be mixed in a composite manner to be used as a catalyst.

Moreover, the method of converting the mixed gas into hydrocarbon to be in contact with the catalyst may be carried out in one stage. However, more preferably, the mixed gas including hydrogen and carbon monoxide generated from biomass as main components thereof is caused to be in contact with the catalyst to convert the gas a into hydrocarbon by the predetermined reaction as a first step, after this step is over, unreacted mixed gas remained in the first step is caused to be in contact with a catalyst equivalent to the aforementioned catalyst to convert the gas a into hydrocarbon by the catalytic reaction as a second step, and further, subsequently, similar steps are carried out as a third step and a fourth step to follow previously determined number of steps to repeatedly carry out predetermined catalytic reaction so that unreacted mixed gas is converted into hydrocarbon and the amount of the mixed gas is reduced in a stepwise manner.

Further, hydrogen obtained by electrolysis of water by the power of recyclable energy other than a biomass may be added to the mixed gas generated from biomass and in this case it becomes possible to significantly improve yield of hydrocarbon per raw material biomass.

An apparatus 1 for producing hydrocarbon from a biomass according to the present embodiment for specifically carrying out the method for producing hydrocarbon from a biomass as explained above includes a biomass gasification unit 101 for generating a mixed gas G including hydrogen and carbon monoxide as main components thereof by supplying a raw material biomass MB, overheated water vapor S for gasification of the raw material biomass MB, fuel biomass FB, and air A for combustion of the fuel biomass FB, a cleaning up means 201 for purifying the mixed gas G generated by the biomass gasification unit 101, a gas tank 301 for temporarily storing the cleaned-up mixed gas G, a pressurization pump 401 for pressurizing the mixed gas G, and a hydrocarbon synthesis unit 501 for converting the pressurized mixed gas G into hydrocarbon, as shown in FIG. 1.

The biomass gasification unit 101 includes an heat insulating room 110 for blocking heat from inside and outside of the room, a gasification reaction chamber 120 provided in the heat insulating room 110, a raw material biomass introduction means 130 for supplying the raw material biomass MB roughly crushed to have a diameter of approximately 2 cm or less into the gasification reaction chamber 120, an overheated water vapor introduction means 140 for supplying the overheated water vapor S into the gasification reaction chamber 120, and a combustion high temperature gas generation unit 150 for supplying a combustion high temperature gas B to a space between the heat insulating room 110 and the gasification reaction chamber 120.

At an appropriate height position in the gasification reaction chamber 120, a porous body 121 having a plurality of through-holes which communicate vertically for partitioning the gasification reaction chamber 120 between upper and lower parts is provided. Moreover, the gasification reaction chamber 120 includes an ash discharging means 122 for discharging ash generated in the gasification reaction chamber 120 outside and a mixed gas exhaust means 123 for emitting the mixed gas G having hydrogen and carbon monoxide as its main components which are generated in the gasification reaction chamber 120 to outside.

The heat insulating room 110 is for blocking heat to and from the room and especially is configured to be enabled to heat inside the heat insulating room 110 and to maintain required temperature, preferably 800° C. or more. The heat insulating room 110 may be configured by a heretofore known heat insulating material and as long as the gasification reaction chamber 120 provided in the heat insulating room 110 can be surrounded, shape or size are appropriately designed. However, a space is provided between an inner surface of the heat insulating room 110 and an external surface of the gasification reaction chamber 120 and the combustion high temperature gas B generated by the combustion high temperature gas generation unit 150 is introduced thereto so that the gasification reaction chamber 120 can be heated from outside of the wall.

To the heat insulating room 110, connecting holes which connect the raw material biomass introduction means 130, the overheated water vapor introduction means 140, the mixed gas exhaust means 123, or ash discharging means 122 to outside of the heat insulating room 110 to communicate inside and outside of the room are formed in a closely connected manner respectively to the raw material biomass introduction means 130, the overheated water vapor introduction means 140, the mixed gas exhaust means 123, ash discharging means 122, or the like to prevent heat from leaking.

Moreover, a combustion high temperature gas inlet 111 and a combustion high temperature gas exhaust 112 are formed to the heat insulating room 110 so that combustion high temperature gas B can be supplied from the combustion high temperature gas generation unit 150 into the heat insulating room 110 or can be emitted from the room.

The gasification reaction chamber 120 is partitioned and surrounded by a thermal conductive wall material, has a gasification space 124 having a predetermined capacity and area inside thereof, and the external surface of the wall material of the gasification reaction chamber 120 is further surrounded by a wall surface of the heat insulating room 110. In the gasification reaction chamber 120, a raw material biomass inlet 125 for supplying the raw material biomass MB from outside into the gasification reaction chamber 120 and an overheated water vapor inlet 126 for supplying the overheated water vapor S from outside into the gasification reaction chamber 120 are formed, which are respectively connected to the raw material biomass introduction means 130 and the overheated water vapor introduction means 140 so that the raw material biomass MB and the overheated water vapor S can be introduced into the gasification reaction chamber 120.

The raw material biomass inlet 125 is formed on an upper part of the gasification reaction chamber 120 and the raw material biomass MB to be introduced from outside into the gasification reaction chamber 120 through the raw material biomass inlet 125, and the raw material biomass is dropped inside the gasification reaction chamber 120 and in the process of dropping, gasification can be carried out.

The overheated water vapor inlet 126 is formed in a lower part of the gasification reaction chamber 120 and the overheated water vapor S to be introduced from outside into the gasification reaction chamber 120 through the overheated water vapor inlet 126 can be introduced as an upward flow in the gasification reaction chamber 120.

Moreover, the gasification reaction chamber 120 has a mixed gas exhaust 127 for emitting the mixed gas G generated inside the chamber from the gasification reaction chamber 120 and an ash exhaust 128 for discharging ash which is, though very little, generated during gasification by the raw material biomass MB and the overheated water vapor S in the gasification reaction chamber 120, each of which is respectively connected to a mixed gas exhaust means 123 and an ash discharging means 122 so that the mixed gas G or the ash generated in the gasification reaction chamber 120 can be emitted outside.

The mixed gas exhaust 127 is formed in an appropriate height position on a side surface of the gasification reaction chamber 120, preferably in a position higher than the height position where the porous body 121 is provided. On the other hand, the ash exhaust 128 is formed on the bottom of the gasification reaction chamber 120 which is lower than the porous body 121 so that in a case where the ash is accumulated, the ash falls by its own weight and is taken outside.

The wall material of the gasification reaction chamber 120 includes a material having thermal conductivity, heat resistance, and thermal shock properties which allows heat to be easily transferred from outside to inside of the gasification reaction chamber 120 and also endures required temperature and change in temperature. Capacity and shape of the gasification space 124 in the gasification reaction chamber 120 may be appropriately designed depending on the required gasification processing throughput. However, the gasification space 124 is designed to have the size and shape which allows the raw material biomass MB which is a target of gasification to exist by an appropriate amount. Surface area inside the gasification reaction chamber 120 may be appropriately designed depending on the required gasification processing throughput.

Inside of the gasification reaction chamber 120 is partitioned in upper and lower parts by the porous body 121 having appropriate thickness which is provided at an appropriate height position in vertical directions. The porous body 121 includes a metal or ceramics which can endure required high temperature, is approximately plate-shaped as a whole, and has a plurality of through-holes which communicate vertically. It is preferable that the size of the through-holes is designed to have a diameter which allows water vapor to pass through without difficulty but does not allow the raw material biomass MB which has not been gasified to pass through. Moreover, the porous body 121 may be provided in a condition slightly inclined than level.

The raw material biomass introduction means 130 connects with the raw material biomass inlet 125 formed to the gasification reaction chamber 120 and includes a pipe of a heat resistant material having a predetermined inner diameter and length which is extended approximately vertically to outside of the heat insulating room 110 through the connecting hole formed to the heat insulating room 110, a screw feeder 131 having an exit connected to the upper edge of the pipe and a screw extended approximately horizontally provided inside the screw feeder 131, and a hopper 132 for supplying the raw material biomass MB to the screw feeder 131.

The screw feeder 131 includes a cylindrical body extended for a predetermined length in approximately horizontal direction, a screw provided inside the cylindrical body in a rotatable manner having approximately same length as the cylindrical body, and an actuator for driving the screw provided on one edge of the screw. In the vicinity of an edge opposite to the side where the actuator is provided, an exit for discharging the raw material biomass MB supplied by the rotation of the screw is formed in the cylindrical body and on an upper part in the vicinity of the actuator in the cylindrical body, an inlet for taking the raw material biomass MB from the hopper 132 into the screw feeder 131 is formed. Of course, the hopper 132 is continued to this inlet.

The overheated water vapor introduction means 140 is connected to the overheated water vapor inlet 126 formed to the gasification reaction chamber 120 and includes a pipe having a heat resistant and water vapor resistant material and having a predetermined inner diameter and length which is extended to outside of the heat insulating room 110 through the communicating hole formed to the heat insulating room 110. It is preferable that a boiler 141 for obtaining overheated water vapor S generated by heating water with the combustion high temperature gas B discharged from the heat insulating room 110 as a heat source is connected on the downstream of the pipe and the overheated water vapor S is previously heated before introduction into the gasification reaction chamber 120 to be the overheated water vapor S. The combustion high temperature gas B which passed out through the boiler 141 is discharged from the chimney 143 via the fan motor 142.

The mixed gas exhaust means 123 is connected to the mixed gas exhaust 127 formed to the gasification reaction chamber 120 and includes a pipe of a heat resistant or corrosion resistant material having a predetermined inner diameter and length which is extended to outside of the heat insulating room 110 through the communication hole formed to the heat insulating room 110. The cleaning up means 201 for purifying the generated mixed gas is connected on the downstream of the pipe.

The ash discharging means 122 is connected to the ash exhaust 128 formed to the gasification reaction chamber 120 and includes a pipe of a heat resistant material having a predetermined inner diameter and length which is extended to outside of the heat insulating room 110 through the communication hole formed to the heat insulating room 110. It is preferable that a valve which can freely open or close the pipe for opening or closing communication between inner and external side of the gasification reaction chamber 120 by the pipe is provided on the downstream of the pipe.

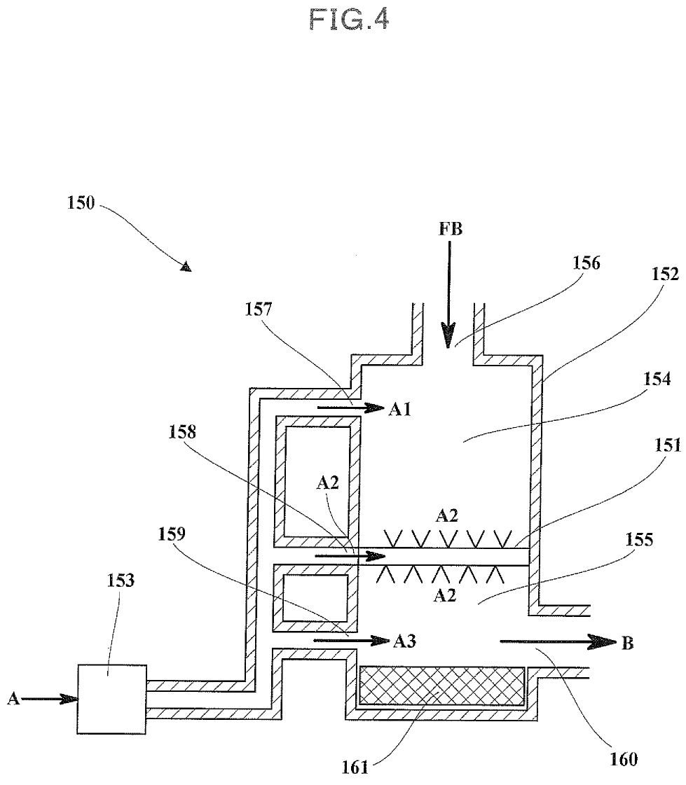

The combustion high temperature gas generation unit 150 includes a combustion furnace 152 formed to have a vertical shape with a fire lattice 151 provided inside at approximate center in height direction thereof and an air preheating device 153 for previously heating an air A to be introduced into the combustion furnace 152, as shown in FIG. 4.

The combustion furnace 152 has an upper combustion room 154 positioned on an upper part of the fire lattice 151 and a bottom combustion room 155 positioned on a lower part of the fire lattice 151. The combustion furnace 152 includes a fuel biomass inlet 156 for supplying a fuel biomass FB roughly crashed on the upper part inside thereof and a first air inlet 157 for supplying an air A1 so that the air for combusting the fuel biomass FB introduced into the upper combustion room 154 is blown into the room. At the approximate center in height direction of the combustion furnace 152, a second air inlet 158 for blowing an air A2 from the fire lattice 151 while introducing the air A2 into the fire lattice 151 provided inside the furnace is formed for more efficiently combusting the fuel biomass FB which falls while burning. In the vicinity of the bottom of the combustion furnace 152, a third air inlet 159 for introducing an air A3 into the bottom combustion room 155 for more completely combusting the combustion high temperature gas B flown down into the bottom combustion room 155 through the fire lattice 151 is formed so that the introduced air A3 is blown in approximately horizontal direction in the combustion furnace 152. A combustion high temperature gas exit 160 for discharging the approximately completely combusted combustion high temperature gas B is formed in a position opposite to the third air inlet 159 on a side wall of the bottom combustion room 155. An ash pit 161 for accumulating ash which is cinder after combustion of the fuel biomass FB is formed on the bottom part of the combustion furnace 152.

The fire lattice 151 is metallic and has a lattice shape having a through path inside thereof through which the air A2 introduced from the second air inlet 158 flows and on both top and bottom surfaces of the lattice-shaped fire lattice 151, a plurality of air nozzles are formed so that the air A2 introduced from the second air inlet 158 is blown out therefrom up-and-down directions.

The air preheating device 153 heat the air A to be introduced into the combustion furnace 152 from the first air inlet 157, the second air inlet 158, or the third air inlet 159 up to 450° C. in advance by use of part of the combustion high temperature gas B generated by the combustion high temperature gas generation unit 150.

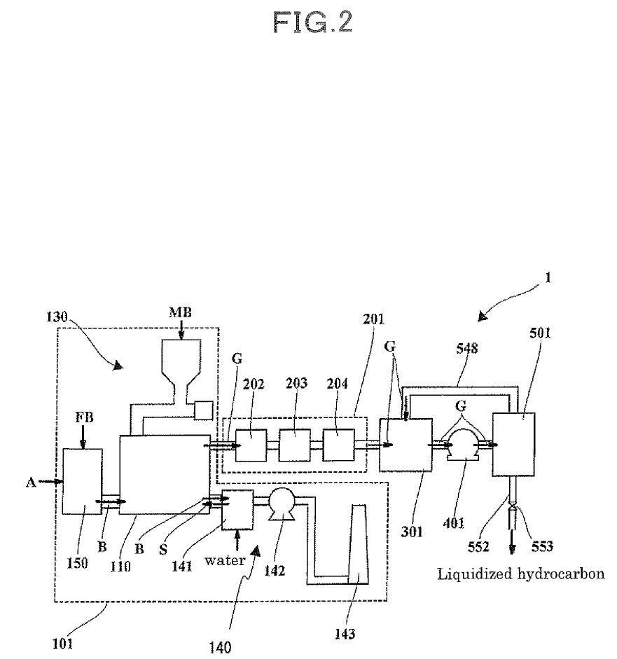

The cleaning up means 201 includes a heat exchanger 202, a cyclone 203, and a water injector 203 as shown in FIG. 2 and they are connected in series to allow the mixed gas G to pass through the heat exchanger 202, the cyclone 203, and the water injector 204 so that ash, soot, tar, or water which is very little but mixed in the mixed gas G which passed through this is removed for purification by the cyclone 203 and the water injector 204 while excess heat is removed by heat exchange in the heat exchanger 202. At the downmost stream of the series of the heat exchanger 202, the cyclone 203, and the water injector 204, a gas tank 301 for temporarily accumulating the purified mixed gas G is connected.

The gas tank 301 is configured to store temporarily the mixed gas G including hydrogen and carbon monoxide as its main components which were purified by passing through the cleaning up means 201 as shown in FIG. 2 and includes a purified mixed gas inlet 302 for supplying the purified mixed gas G into inside, a mixed gas exit 303 for transferring the mixed gas G to a pressurization pump 401 connected on the downstream of the gas tank 301, and an inlet for reintroduction of mixed gas 304 for supplying unreacted mixed gas G which finally remained unreacted in a hydrocarbon synthesis unit 501 into the gas tank 301 again.

The pressurization pump 401 is connected to the mixed gas exit 303 of the gas tank 301 arranged just upstream and is configured to pressurize the mixed gas G up to required pressure while allowing the mixed gas G which was temporarily stored to flow down from the gas tank 301 and feeds the mixed gas G pressurized to the required pressure to the hydrocarbon synthesis unit 501 connected on just downstream of the pressurization pump 401, as shown in FIG. 2.

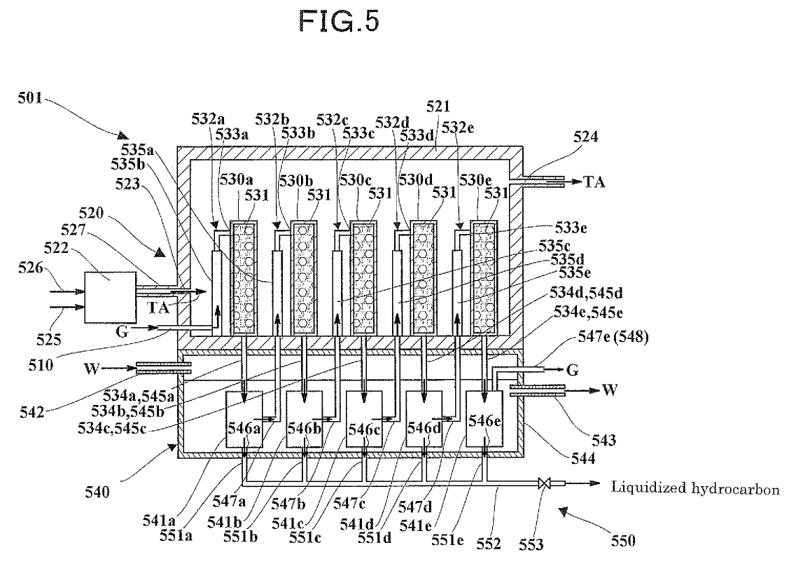

The hydrocarbon synthesis unit 501 includes, as shown in FIG. 5, a pressurized mixed gas inlet 510 for supplying the mixed gas G pressurized up to the required pressure by the pressurization pump 401, a temperature adjustment means 520 for adjusting the introduced mixed gas G to appropriate temperature, and a catalyst 531 for obtaining hydrocarbon as a product by the predetermined catalytic reaction as explained above by use of the mixed gas G maintained to the appropriate temperature by the temperature adjustment means 520 while being pressurized by the pressurization pump 401, as a reactant and has a reaction chamber 530 for the predetermined catalytic reaction of contacting the mixed gas G with the catalyst 531 under appropriate temperature and pressure, a liquidization means 540 for liquidizing the hydrocarbon generated by the catalytic reaction, and a collection means 550 for collecting the liquidized hydrocarbon liquidized by the liquidization means 540.

The temperature adjustment means 520 includes constant temperature room 521 and a constant temperature room temperature adjustment unit 522 for adjusting temperature in the constant temperature room 521. The constant temperature room 521 is partitioned and surrounded by an heat insulating material to have a predetermined volume capacity and has a pressurized mixed gas inlet 510 for supplying the mixed gas G inside thereof pressurized by the pressurization pump 401, a temperature-controlled air inlet 523 for supplying a temperature-controlled air TA which is controlled to have a required temperature by the constant temperature room temperature adjustment unit 522, and a temperature-controlled air exhaust 524 for discharging the temperature-controlled air TA which was introduced into the constant temperature room 521 and flew down in the constant temperature room 521. In the constant temperature room 521, a plurality of reaction chambers 530 are provided. That is, in the present example, first to fifth reaction chambers 530a, 530b, 530c, 530d, and 530e, which are equivalent to each other, are provided and these first to fifth reaction chambers 530a, 530b, 530c, 530d, and 530e can be maintained in the required temperature holistically.

The constant temperature room temperature adjustment unit 522 includes a high temperature gas introduction line 525 for supplying a high temperature gas, an air introduction line 526, and a temperature-controlled air feeding line 527. The temperature-controlled air feeding line 527 generates the temperature-controlled air TA by bringing an air introduced from the air introduction line 526 into direct or indirect contact with the high temperature gas introduced from the high temperature gas introduction line 525 to control the air to have required temperature and feeds the temperature-controlled air TA into the constant temperature room 521. Inside temperature of the constant temperature room 521 is maintained to be the required temperature by feeding an appropriate amount of the temperature-controlled air TA with appropriate temperature generated by the constant temperature room temperature adjustment unit 522 into the constant temperature room 521 and at the same time by discharging the temperature-controlled air TA from the constant temperature room 521 to flow. Thus, the mixed gas G to be introduced into the constant temperature room 521 or temperature of the first to fifth reaction chambers 530a, 530b, 530c, 530d, and 530e can be maintained to the required temperature. Here, the combustion high temperature gas B discharged from the biomass gasification unit or the like may be used as the high temperature gas.

The first to fifth reaction chambers 530a, 530b, 530c, 530d, and 530e respectively has first to fifth mixed gas inlets 533a, 533b, 533c, 533d, and 533e respectively connected to first to fifth mixed gas introduction lines 532a, 532b, 532c, 532d, and 532e for setting the mixed gas G pressurized to the predetermined pressure to have the predetermined temperature and for supplying the mixed gas G into first to fifth reaction chambers 530a, 530b, 530c, 530d, and 530e; the catalysts 531 provided in the first to fifth reaction chambers 530a, 530b, 530c, 530d, and 530e for causing the mixed gas G introduced into the first to fifth reaction chambers 530a, 530b, 530c, 530d, and 530e to carry out catalytic reaction; and discharge lines 534a, 534b, 534c, 534d, and 534e for discharging outside the unreacted mixed gas G which flew down the first to fifth reaction chambers 530a, 530b, 530c, 530d, and 530e or hydrocarbon generated by the catalytic reaction. Here, the FT catalyst or the above-mentioned composite catalyst may be used as the catalyst 531.

The first to fifth mixed gas introduction lines 532a, 532b, 532c, 532d, and 532e have first to fifth temperature adjustment units 535a, 535b, 535c, 535d, and 535e for adjusting the mixed gas G to have the required temperature by bringing the mixed gas G into indirect contact with the temperature-controlled air TA introduced into the constant temperature room 521 before the mixed gas G is introduced into the reaction chamber 530 so that the mixed gas G can be adjusted to the required temperature before the gas is respectively introduced into the first to fifth reaction chambers 530a, 530b, 530c, 530d, and 530e.

The first to fifth reaction chambers 530 are connected in series to gradually convert the unreacted mixed gas G which flows from upstream side to the downstream side into hydrocarbon and at the same time to reduce the amount of the unreacted mixed gas G.

In the hydrocarbon synthesis unit 501 of the present embodiment, the pressurized mixed gas inlet 510 is connected to the first reaction chamber 530a through the first mixed gas introduction line 532a and the first reaction chamber 530a is connected to the second reaction chamber 530b through the first liquidization chamber 541a connected in the downstream of the first reaction chamber 530a and the second mixed gas introduction line 532b connected in the further downstream thereof. Then, the second reaction chamber 530b is connected to the third reaction chamber 530c through the second liquidization chamber 541b connected in the downstream of the second reaction chamber 530b and the third mixed gas introduction line 532c connected in the further downstream thereof. Similarly, the third reaction chamber 530c is connected to the fourth reaction chamber 530d through the third liquidization chamber 541c connected in the downstream of the third reaction chamber 530c and the fourth mixed gas introduction line 532d connected in the further downstream thereof, and the fourth reaction chamber 530d is connected to the fifth reaction chamber 530e through the fourth liquidization chamber 541d connected in the downstream of the fourth reaction chamber 530d and the fifth mixed gas introduction line 532e connected in the further downstream thereof. Thus, the five-staged first to fifth reaction chambers 530a, 530b, 530c, 530d, and 530e are set. Here, although the number of stages of the reaction chambers is set to five, it is needless to say that the stage is not limited to five and the number of stages may be designed appropriately.

On the downstream of the exhaust line 534e of the fifth reaction chamber 530e which discharges hydrocarbon and the unreacted mixed gas G generated in the fifth reaction chamber 530e, the fifth liquidization chamber 541e is connected.

The liquidization means 540 includes a cooling water introduction line 542 for supplying a cooling water W and a cooling water discharging line 543 for discharging the cooling water W and has a cooling bath 544 which stores the cooling water W introduced inside. In the cooling bath 544, the first liquidization chamber 541a, the second liquidization chamber 541b, the third liquidization chamber 541c, the fourth liquidization chamber 541d, and the fifth liquidization chamber 541e are provided and these first to fifth liquidization chambers 541a, 541b, 541c, 541d, and 541e are holistically cooled down by the cooling water W which flows down in the cooling bath 544.

The first to fourth liquidization chambers 541a, 541b, 541c, and 541d are configured to be equivalent to each other and each of them respectively has introduction lines 545a, 545b, 545c, and 545d for supplying the unreacted mixed gas G and generated hydrocarbon flown down from the first to fourth reaction chambers 530a, 530b, 530c, and 530d connected to just above the stream; liquidized hydrocarbon exhausts 546a, 546b, 546c, and 546d for discharging the hydrocarbon cooled and liquidized in the first to fourth liquidization chambers 541a, 541b, 541c, and 541d; and cooled mixed gas exhausts 547a, 547b, 547c, and 547d for discharging the mixed gas G which is gasified though being cooled to the mixed gas introduction lines 532b, 532c, 532d, and 532e connected to just down the stream.

The fifth liquidization chamber 541e includes the introduction line 545e for supplying the unreacted mixed gas G and generated hydrocarbon flown down from the fifth reaction chamber 530e connected to just above the upstream; the liquidized hydrocarbon exhaust 546e for discharging the hydrocarbon cooled down and liquidized in the fifth liquidization chamber 541e; and the final unreacted mixed gas exhaust 547e for discharging the gasified mixed gas G which finally remained unreacted though being cooled. The final unreacted mixed gas exhaust 547e is connected to the mixed gas reintroduction inlet 304 of the gas tank 301 via a circulation line 548 to collect the finally unreacted mixed gas G into the gas tank 301 again and to circulate the gas.

The liquidized hydrocarbon liquidized by the liquidization means 540 is collected by a collection means 550 configured to have a collecting pipe shape. The collection means 550 includes a first collection pipe 551a, a second collection pipe 551b, a third collection pipe 551c, a fourth collection pipe 551d, and a fifth collection pipe 551e respectively connected to the liquidized hydrocarbon exhausts 546a, 546b, 546c, 546d, and 546e of the first to fifth liquidization chambers 530a, 530b, 530c, 530d, and 530e; a liquidized hydrocarbon extraction pipe 552 to which these first to fifth collection pipes 551a, 551b, 551c, 551d, and 551e are connected; and a valve 553 provided in downmost stream of the liquidized hydrocarbon extraction pipe 552, is configured to have a collecting pipe shape as a whole, and the liquidized hydrocarbon can be appropriately taken out by opening and closing control of the valve 553.

As described above, since the apparatus for producing hydrocarbon from a biomass of the present invention is configured to cause catalytic reaction of unreacted mixed gas gradually by connecting a plurality of reaction chambers in series, hydrocarbon can be obtained with high yield even though it is relatively low pressured.

Moreover, since the present invention is configured to maintain temperature in the reaction chamber by providing the reaction chamber in the constant temperature room which temperature can be controlled easily, it becomes possible to easily maintain temperature by a simple configuration without getting the apparatus complexed. Moreover, maintenance of the apparatus is easy, the apparatus is suitable for practical use, and operational reliability as a whole of the apparatus for producing hydrocarbon from a biomass is improved.

Further, in the present invention, The biomass gasification unit using reaction between a raw material biomass and overheated water vapor by supplying heat from outside through a wall enables to gasify the biomass with high yield and to obtain stable production of a mixed gas including hydrogen and carbon monoxide as its main components.

Further, since hydrogen obtained by electrolysis of water by a recyclable energy is added to the mixed gas including hydrogen and carbon monoxide as its main components which are obtained from the biomass gasification unit, yield of hydrocarbon from the raw material biomass can be significantly increased.

The method and the apparatus 1 for producing hydrocarbon from a biomass of the present invention is configured as explained above. However, scope of the present invention is not restricted within the embodiments in this Description.

### INDUSTRIAL APPLICABILITY

As described above, the method of the present invention provides a method for producing hydrocarbon from a biomass using hydrogen and carbon monoxide generated by use of a biomass such as grass or a tree as a raw material as a reactant and enables to synthesize liquid or gas hydrocarbon fuel as a product with a high yield while being small-sized and low pressured, and provides an apparatus thereof.

1. A method for producing hydrocarbon from a biomass comprising, heating a powder or chipped raw material biomass to 800° C. or more, contacting the biomass with water vapor of 800° C. or more to generate a mixed gas including hydrogen and carbon monoxide as main components thereof, contacting the mixed gas with a predetermined catalyst under a predetermined temperature and a predetermined pressure to convert the mixed gas into hydrocarbon.

2. The method for producing hydrocarbon from a biomass according to claim 1, wherein the predetermined pressure is lower than 3 MPa.

3. The method for producing hydrocarbon from a biomass according to either claim 1 or 2,
wherein the catalyst has a single body or a compound of one selected from iron and copper or both of them as a basic catalyst, a single body or a compound of one or more substances selected from magnesium, calcium, cobalt, nickel, potassium, and sodium which is added as a assistant catalyst, and one or more substances selected from zeolite, alumina, and silica.

4. The method for producing hydrocarbon from a biomass according to any one of claims 1 to 3,
wherein the catalytic reaction is a chemical reaction expressed by the following chemical equation formula.
CO+2H₂→n⁻¹(CH₂)ₙ+H₂O

5. The method for producing hydrocarbon from a biomass according to any one of claims 1 to 4,
wherein a step of bringing the mixed gas into contact with the catalyst to convert the gas into hydrocarbon by the predetermined catalytic reaction and a step bringing unreacted mixed gas remained in a previous step into contact with a catalyst equivalent to the catalyst so that the gas is converted into hydrocarbon by the catalytic reaction is set to include predetermined number of steps to repeatedly carry out the predetermined catalytic reaction to the mixed gas so that the unreacted mixed gas is gradually converted into hydrocarbon while the amount of the mixed gas is reduced.

6. The method for producing hydrocarbon from a biomass according to any one of claims 1 to 5,
wherein hydrogen obtained by electrolysis of water by the power of a recyclable energy other than a biomass is added to the mixed gas including hydrogen and carbon monoxide as its main components which are generated by heating the raw material biomass to 800° C. or more and at the same time bringing the biomass into contact with water vapor of 800° C. or more enables to improve yield of hydrocarbon per raw material biomass.

7. An apparatus for producing hydrocarbon from a biomass, comprising,
a biomass gasification unit for generating a mixed gas including hydrogen and carbon monoxide as main components thereof using a biomass as a raw material and fuel;
a pressurization means for pressurizing the mixed gas generated by the biomass gasification unit;
a temperature adjustment means for adjusting the mixed gas to have an appropriate temperature;
a catalyst for obtaining hydrocarbon as a product by causing a predetermined catalytic reaction using the mixed gas which is pressurized by the pressurization means while maintained in the appropriate temperature by the temperature adjustment means as a reactant;
a reaction chamber in which the catalyst is provided and the mixed gas which is appropriately pressurized and has appropriate temperature is brought into contact with the catalyst to cause a predetermined catalytic reaction;
a liquidization means for liquidizing the hydrocarbon generated by the catalytic reaction; and
a collection means for collecting the liquidized hydrocarbon liquidized by the liquidization means.

8. An apparatus for producing hydrocarbon from a biomass according to claim 7,
wherein the reaction chamber includes an inlet for supplying the mixed gas and an exhaust for discharging the hydrocarbon generated by the catalytic reaction in the reaction chamber and the unreacted mixed gas,
wherein the catalyst is provided on a route from the inlet to the exhaust.

9. An apparatus for producing hydrocarbon from a biomass according to either claim 7 or 8,
wherein the reaction chamber has the inlet for supplying the mixed gas in an upper part thereof, has the exhaust for discharging the hydrocarbon generated by the catalytic reaction of the mixed gas in the reaction chamber and the unreacted mixed gas in an lower part of the chamber, and the catalyst is provided on a route from the inlet to the exhaust, a plurality of the reaction chambers are provided, the inlet of the reaction chamber positioned at the uppermost stream connects to the biomass gasification unit so that the mixed gas generated by the biomass gasification unit can be introduced, and the exhaust of the reaction chamber positioned at the uppermost stream communicates with the inlet of the reaction chamber positioned on immediate downstream, and hereinafter an exhaust of the reaction chamber positioned on immediate upper stream is connected to the inlet of the reaction chamber positioned on immediate downstream.

10. An apparatus for producing hydrocarbon from a biomass according to claim 9,
wherein the liquidization means includes a liquidization chamber having the inlet on the upper stream side and an exhaust for discharging the unreacted mixed gas and an extraction port for extracting liquidized hydrocarbon on the lower stream side and a cooling means for cooling down the liquidization chamber, wherein the inlet is connected to the exhaust of the reaction chamber positioned on the upper stream side of the liquidization chamber and the exhaust is connected to the inlet of the reaction chamber positioned on the lower stream side of the liquidization chamber.

11. An apparatus for producing hydrocarbon from a biomass according to claim 10,
wherein the collection means for collecting the liquidized hydrocarbon includes a pipeline connecting with each extraction ports of the plurality of liquidization chambers,
wherein the pipeline can be freely opened or closed by a valve provided on a route thereof.

12. An apparatus for producing hydrocarbon from a biomass according to any one of claims 7 to 11,
wherein a temperature adjustment means for adjusting temperature of the mixed gas to be the predetermined temperature before introduction of the mixed gas into the reaction chamber intervenes on the upper stream side of the inlet for supplying the mixed gas into the reaction chamber.

13. An apparatus for producing hydrocarbon from a biomass according to any one of claims 7 to 12,
wherein the reaction chamber includes an air inlet for supplying air controlled to have a predetermined temperature by a constant room temperature adjustment means and an air exhaust for emitting air and is provided in a constant temperature room which is partitioned and surrounded by an heat insulating material.

14. An apparatus for producing hydrocarbon from a biomass according to any one of claims 7 to 13,
wherein the catalyst has a single body or a compound of one selected from iron and copper or both of them as a basic catalyst and at the same time a single body or a compound of one or more substances selected from magnesium, calcium, cobalt, nickel, potassium, and sodium is added as a backup catalyst or assistant catalyst thereto, while one or more substances selected from zeolite, alumina, and silica are supported.

15. An apparatus for producing hydrocarbon from a biomass according to any one of claims 7 to 14,
wherein the biomass gasification unit includes an heat insulating room which is partitioned and surrounded by an heat insulating material, a gasification reaction chamber having a raw material biomass introduction means partitioned and surrounded by a thermally conductive wall material in the heat insulating room for supplying the raw material biomass roughly crushed to have a diameter of approximately 2 cm or less inside and an overheated water vapor introduction means for supplying overheated water vapor inside, and a combustion high temperature gas generation unit for supplying a combustion high temperature gas in a space between the heat insulating room and the gasification reaction chamber,
wherein the raw material biomass and the overheated water vapor introduced into the gasification reaction chamber are heated by the combustion high temperature gas supplied from the combustion high temperature gas generation unit to the space between the heat insulating room and the gasification reaction chamber via the thermally conductive wall material of the gasification reaction chamber to cause the raw material biomass and the overheated water vapor to react endothermically so that the mixed gas including hydrogen and carbon monoxide as main components thereof is generated.

16. An apparatus for producing hydrocarbon from a biomass according to any one of claims 7 to 15,
wherein the combustion high temperature gas generation unit generates a combustion high temperature gas of 800° C. or more by perfectly combusting a fuel biomass and supplies the combustion high temperature gas to the space between the heat insulating room and the gasification reaction chamber.
